SEQUENCE LISTING (1 sequence)

SEQ ID NO: 1 - protein, 153 aa, synthetic construct
MRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

(19) United States
(12) Patent Application Publication
(10) Pub. No.: US 2025/0388647 A1
(43) Pub. Date: Dec. 25, 2025

(54) THERAPIES FOR MUCOPOLYSACCHARIDOSES III

(71) Applicants: Lundquist Institute for Biomedical Innovation at Harbor-UCLA Medical Center, Torrance, CA (US); Cure Sanfilippo Foundation, Columbia, SC (US)

(72) Inventors: Lynda E. Polgreen, Torrance, CA (US); Cara O'Neill, Columbia, SC (US)

(21) Appl. No.: 19/244,857

(22) Filed: Jun. 20, 2025

Related U.S. Application Data
(60) Provisional application No. 63/662,147, filed on Jun. 20, 2024.

(51) Int. Cl. C07K 14/715; A61K 38/00; A61P 1/12

(52) U.S. Cl. CPC C07K 14/7155; A61P 1/12; A61K 38/00

(57) ABSTRACT

Methods for treating a mucopolysaccharidosis III (MPS III), also known as Sanfilippo syndrome, in a patient in need thereof are provided. In one aspect, the method comprises administering to the patient an effective amount of a polypeptide comprising human IL-1Ra, or a sequence having at last 85% sequence identity to the human IL-1Ra.

### CROSS-REFERENCE TO REPLATED APPLICATIONS

This application claims the benefit under 35 U.S.C. § 119 (e) to U.S. Provisional Application Ser. No. 63/662,147, filed on Jun. 20, 2024, which is hereby incorporated by reference in its entirety.

### REFERENCE TO AN ELECTRONIC SEQUENCE LISTING

The contents of the electronic sequence listing “0WVR-385736-US.xml” (size: 2,078 bytes, created Jun. 20, 2025), is herein incorporated by reference in its entirety.

### BACKGROUND

Mucopolysaccharidoses (MPS) is caused by the absence or malfunctioning of lysosomal enzymes needed to break down glycosaminoglycans (GAGs). These long chains of sugar carbohydrates occur within the cells that help build bone, cartilage, tendons, corneas, skin and connective tissue. GAGs (formerly called mucopolysaccharides) are also found in the fluids that lubricate joints.

Individuals with MPS either do not produce enough of one of the eleven enzymes required to break down these sugar chains into simpler molecules, or they produce enzymes that do not work properly. Over time, these GAGs collect in the cells, blood and connective tissues. The result is permanent, progressive cellular damage which affects appearance, physical abilities, organ and system functioning. Most MPS affect the central nervous system of children and result in severe progressive neurodegenerative decline eventually leading to handicap and death.

MPS III, also known as Sanfilippo syndrome, is marked by severe neurological symptoms. Sanfilippo syndrome is a fatal childhood neurodegenerative disorder characterized by regression in development, loss of speech, disordered sleep and movement, pain, and intensifying neurobehavioral symptoms, such as hyperactivity, agitation, destructiveness, distress/screaming, and social disengagement. Symptom onset often begins around age three to five years, followed by an unremitting disease course culminating in death in the second or third decade.

Sanfilippo syndrome is one of a group of seven mucopolysaccharidosis disorders defined by deficiency in lysosomal enzymes critical to degrading glycosaminoglycans (GAG). Accumulating GAG trigger pathological cascades and cellular dysfunction that lead to worsening clinical disease. Enzyme restorative approaches have been successful in attenuating or halting disease progression in most other forms of MPS, but none have been approved for any of the MPS III subtypes.

As the pursuit of enzyme restoration continues, there is urgency to palliate symptoms that cause suffering for the majority of the Sanfilippo community. While clinical endpoints of most MPS III clinical trial programs have focused on neurocognitive decline, recent publications have indicated that pain, disordered sleep and movement, and neurobehavioral symptoms are among the most important symptoms to the Sanfilippo community to treat.

### SUMMARY

In one aspect, a method for treating a mucopolysaccharidosis III (MPS III) in a patient in need thereof is provided. The method comprises administering to the patient an effective amount of a polypeptide comprising human IL-1Ra or a sequence having at least 85% sequence identity to the human IL-1Ra.

In some embodiments, the polypeptide comprises anakinra. In some embodiments, the polypeptide consists of anakinra. In some embodiments, the polypeptide is administered at 50 mg to 500 mg daily. In some embodiments, the polypeptide is administered at 100 mg or 200 mg daily. In some embodiments, the polypeptide is administered once daily. In some embodiments, the administration is subcutaneous. In some embodiments, the polypeptide is administered for 8 weeks or more. In some embodiments, the polypeptide is administered for 16 weeks or more. In some embodiments, wherein the polypeptide is administered for 36 weeks or more. In some embodiments, the method comprises administering 100 mg or 200 mg of anakinra once daily. In some embodiments, the MPS III is MPS IIIA, MPS IIIB, MPS IIIC, or MPS IIID. In some embodiments, the MPS III is MPS IIIA, MPS IIIB or MPS IIIC. In some embodiments, the patient has at least one of: a movement disorder; fatigue, pain, a Sanfilippo behavior, a sleep problem, loose stool, hyperactivity, communication problems, or behavioral problem. In some embodiments, the patient has a child Sleep Health Questionnaire (CSHQ) Total score≥41. In some embodiments, the patient has a Sanfilippo Behavior Rating Scale (SBRS) Cluster or Domain score≥−2 SD of mean for age group. In some embodiments, the patient has significant MPS III related central nervous system (CNS) impairment or behavioral disturbance. In some embodiments, the patient has a Non-communicating Children's Pain Checklist—Revised (NCCPC-R) Total Score of ≥7. In some embodiments, the patient is an adult (18 years or older). In some embodiments, the patient is a minor (17 years or younger).

### BRIEF DESCRIPTION OF DRAWINGS

FIG. 1 is a flowchart for a Phase 1/2, eight-week open-label study, followed by a 28-week open-label extension.

FIG. 2 is graphs showing multi-domain responder index (MDRI) for comparison of change during (A) 8 weeks, (B) 16 weeks, and (C) 36 weeks of treatment to minimal clinically important difference (MCID).

FIG. 3 is a graph showing change in the Individual Clinical Response (ICR) before, during and after treatment with anakinra.

FIG. 4 is a graph showing change in the Sanfilippo Behavior Rating Scale (SBRS) individual clusters and domains before, during and after treatment with anakinra.

FIG. 5A is flow cytometry plots of whole blood stained with a specific antibody to identify monocyte.

FIG. 5B is flow cytometry plots of whole blood stained with a specific antibody to identify monocyte

FIG. 5C is graphs showing changes in CD4+ T cells (e.g., T helper, T regulatory cells), CD8+ T cells (e.g., cytotoxic T cells), monocytes, and B cells over 16 weeks of treatment with anakinra, compared to the multi-domain responder index (MDRI).

FIG. 6A is a graph showing CSHQ total score in “Sleep Disturbances” subgroup.

FIG. 6B is a graph showing total number of Sleep Problems in the CSHQ in “Sleep Disturbances” subgroup.

FIG. 6C is a graph showing LS severity in “Loose Stool” subgroup.

FIG. 6D is a graph showing SBRS mood/anger/aggression in “Behavioral Problems” subgroup.

FIG. 7A is a graph showing minimal clinically important difference (MCID) changes in CSHQ total score in “Sleep Disturbances” subgroup.

FIG. 7B is a graph showing minimal clinically important difference (MCID) changes in total number of Sleep Problems in the CSHQ in “Sleep Disturbances” subgroup.

FIG. 7C is a graph showing minimal clinically important difference (MCID) changes in LS severity in “Loose Stool” subgroup.

FIG. 7D is a graph showing minimal clinically important difference (MCID) changes in SBRS mood/anger/aggression in “Behavioral Problems” subgroup.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment. In a preferred embodiment, the patient is human. In a more preferred embodiment, the patient is children or adolescent.

### Treatment for Mucopolysaccharidoses III (MPS III)

In one aspect, the present disclosure provides methods of treating Mucopolysaccharidoses III (MPS III). For the treatment method provided herein, a polypeptide comprising human Interleukin 1 receptor antagonist (IL-1Ra) or a sequence having at least 80%, 85%, 90%, 95%, 97%, 98%, or 99% sequence identity to human IL-1Ra may be used.

In some embodiments, the polypeptide comprises anakinra. In some embodiments, the polypeptide is anakinra. Anakinra, sold under the brand name Kineret, is a biopharmaceutical medication used to treat rheumatoid arthritis, cryopyrin-associated periodic syndromes, familial Mediterranean fever, and Still's disease. Anakinra is administered by subcutaneous injection.

Anakinra differs from the sequence of Interleukin 1 receptor antagonist by one methionine amino acid added to its N-terminus. The amino acid sequence of anakinra is shown in the table below (SEQ ID NO:1).

Amino acid sequence of anakinra (SEQ ID NO: 1)

MRPSGRKSSK MQAFRIWDVN QKTFYLRNNQ LVAGYLQGPN
VNLEEKIDVV PIEPHALFLG IHGGKMCLSC VKSGDETRLQ
LEAVNITDLS ENRKQDKRFA FIRSDSGPTT SFESAACPGW
FLCTAMEADQ PVSLINMPDE GVMVTKFYFQ EDE

In some embodiments, the polypeptide comprising human Interleukin 1 receptor antagonist (IL-1Ra), or anakinra, is administered for about a week, two weeks, three weeks, four weeks, eight weeks, sixteen weeks, thirty two weeks, thirty six weeks, forty four weeks or longer. In some embodiments, the duration of the drug administration lasts about two months, three months, four months, sixteen weeks, five months, six months, seven months, eight months, nine months, thirty six weeks, ten months, eleven months, twelve months, thirteen months, fourteen months, fifteen months or longer.

The present disclosure also provides pharmaceutical compositions suitable for administration, such as oral, sublingual, buccal, intranasal, intrathecal, intravenous, intramuscular, transdermal, or intraperitoneal administration. For instance, for intranasal administration, the anakinra and/or other agents may be retained in the submucous space of the nose, cross the arachnoid membrane, and enter into the central nervous system via the olfactory pathways. In some embodiments, the anakinra and/or other agents may be administered subcutaneously. In some embodiments, a transport moiety complex is included to facilitate transport of the agent to the CNS, thereby improving response time and minimizing exposure of peripheral tissues to the active agents.

In some embodiments, the MPS III is MPS IIIA, MPS IIIB, MPS IIIC, or MPS IIID. For example, MPS III may be MPS IIIA, MPS IIIB or MPS IIIC. In some embodiments, the MPS III is Sanfilippo syndrome.

In some embodiments, the patient may have one or more symptoms of: a movement disorder; fatigue, pain, a Sanfilippo behavior, a sleep problem, loose stool or other digestive problem, hyperactivity, communication problems, behavioral problem, central nervous system (CNS) impairment, seizure. In some embodiments, the patient has a child Sleep Health Questionnaire (CSHQ) Total score≥41. In some embodiments, the patient has a Sanfilippo Behavior Rating Scale (SBRS) Cluster or Domain score≥−2 SD of mean for age group. In some embodiments, the patient has significant MPS III related central nervous system (CNS) impairment or behavioral disturbance. In some embodiments, the patient has a Non-communicating Children's Pain Checklist—Revised (NCCPC-R) Total Score of ≥7.

In some embodiments, the patient is a minor. In some embodiments, the patient is younger than 15, 16, 17, 18, or 19 years old. In some embodiments, the patient is older than 3, 4, 5, or 6 years old. In other embodiments, the patient is an adult.

In some embodiments, the patient of the present disclosure has abnormal biomarker levels of neuroinflammation, such as CD68, lysosomal enlargement Lamp1, IBA1, and translocator protein (TSPO) (in microglia) and/or GFAP (in astrocyte). In some embodiments, the patient of the present disclosure has abnormal biomarker levels of the MPS, in particular MPSIII, for instance glycosaminoglycans (GAGs), such as heparin sulfate (HS).

In some embodiments, the administration of anakinra decreases the serum level or cerebral spinal fluid (CSF) level of the biomarkers of neuroinflammation by about 10%, about 20%, about 30%, about 40%, about 50%, about 60%, about 70%, about 80%, or about 90%. In some embodiments, the administration of anakinra decreases the serum level or CSF level of the biomarkers of MPS by about 10%, about 20%, about 30%, about 40%, about 50%, about 60%, about 70%, about 80%, or about 90% in comparison with a control. The control can be a patient without treatment, a patient that has received placebo treatment, or the same patient before anakinra administration.

In some embodiments, the administration of the polypeptide or anakinra alleviates the symptoms of MPS III, for example, one or more of behavioral problem, mood, anger, aggression, social or emotional dysfunction, lack of fear, executive dysfunction, movement problem, orality, sleep problems, or loose stool. In some embodiments, the administration of the polypeptide or anakinra alleviates the neuropathophysiological condition such as sleep problem, aggressive behavior, hyperactivity, seizure, deafness or loss of vision by at least 10%, at least 20%, at least 30%, at least 40%, at least 50%, at least 60%, at least 70%, at least 80%, at least 90% or 100% in comparison with a control. The control can be a patient without treatment, a patient that has received placebo treatment, or the same patient before anakinra administration.

### Dosing

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. In some embodiments a dosage of between 2 and 8 mg/kg may be appropriate. In some embodiments a dosage of about 4 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound described herein administered per dose or per day. Daily dosage of the polypeptide or anakinra may be between about 1 mg and 4,000 mg, between about 2,000 to 4,000 mg/day, between about 1 to 2,000 mg/day, between about 1 to 1,000 mg/day, between about 10 to 500 mg/day, between about 1 to 200 mg/day, between about 10 to 200 mg/day, between about 20 to 500 mg/day, between about 50 to 300 mg/day, between about 75 to 200 mg/day, between about 15 to 150 mg/day or between about 20 to 500 mg/day. In some embodiments, the daily dosage of the polypeptide or anakinra may be about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, about 140 mg/day, about 150 mg/day, about 160 mg/day, about 170 mg/day, about 180 mg/day, about 190 mg/day, about 200 mg/day, about 250 mg/day, about 300 mg/day, or about 500 mg/day. In some embodiments, the daily dosage of seladelpar may be about 100 mg/day or about 200 mg/day.

The compounds of the present application or the compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are well known in cancer chemotherapy, and are frequently alternated with resting periods of about 1 to 28 days, commonly about 7 days or about 14 days, between cycles. The treatment cycles, in other embodiments, may also be continuous.

In a particular embodiment, the method comprises administering to the subject an initial daily dose of about 1 to 800 mg of a compound described herein and increasing the dose by increments until clinical efficacy is achieved. Increments of about 5, 10, 25, 50, or 100 mg can be used to increase the dose. The dosage can be increased daily, every other day, twice per week, or once per week.

In some embodiments, seladelpar or pharmaceutically acceptable salt thereof may be administered in an amount equivalent to 10 mg/day of seladelpar.

To increase the contact time and targeting to the olfactory nerves, formulation of a pharmaceutically active agent-transport moiety with a biocompatible adhesive or a delivery device can be prepared. The formulation may be in the form of a cream, liquid, spray, powder, or suppository which can be administered intranasally using a suitable applicator. Processes for preparing pharmaceuticals in these vehicles can be found throughout the literature. The formulation can be applied using any convenient method or device such as a spray device, metered dose applicator for cream, suppository suitable for intranasal insertion, and the like. The formulation can also include a bioadhesive agent, for example, a mucoadhesive agent. The mucoadhesive agent permits a close and extended contact of the composition, or the drug released from said composition, with mucosal surface by promoting adherence of said composition or drug to the mucosa. The mucoadhesive agent is preferably a polymeric compound, such as preferably, a cellulose derivative but it may be also a natural gum, alginate, pectin, or such similar polymer. A preferred cellulose derivative is hydroxypropyl methylcellulose, commercially available from Dow Chemical Co. The mucoadhesive agent can be present in from about 5 to about 25%, by weight, preferably in from about 10 to about 15% and most preferably about 10%.

Bioadhesive microparticles or nanoparticles can constitute still another component of the intranasal formulations suitable for use in the present disclosure. The bioadhesive particles include derivatives of cellulose such as hydroxypropyl cellulose and polyacrylic acid and can provide sustained release of the pharmaceutically active agents for an extended period of time (possibly days) once they are placed in the appropriate formulation. A formulation comprising bioadhesive particles can provide a multi-phase liquid or semi-solid preparation which does not seep from the nose. The microparticles or nanoparticles cling to the nasal epithelium and can release the drug over extended period of time, for example, for several hours or more.

The biocompatible adhesives can include viscosity enhancers such as methylcellulose, sodium carboxymethylcellulose, chitosan, carbopol 934P and Pluronic 127. Thermogelling agents such as ethyl(hydroxyethyl) cellulose and Pluronic 127 can also be used to advantage. Thermogelling agents are liquid at room temperature and below, but at physiological temperatures (e.g., 32-37° C.), the viscosity of the solution increases such that the solution becomes a gel.

Pharmaceutical compositions may be formulated in combination with any suitable pharmaceutical vehicle, excipient or carrier that would commonly be used in this art, such as saline, dextrose, water, glycerol, ethanol, other therapeutic compounds, and combinations thereof. As one skilled in this art would recognize, the particular vehicle, excipient or carrier used will vary depending on the patient and the patient's condition, and a variety of modes of administration would be suitable for the compositions of the invention, as would be recognized by one of ordinary skill in this art.

Suitable nontoxic pharmaceutically acceptable excipients for use in the compositions of the present invention will be apparent to those skilled in the art of pharmaceutical formulations and examples are described in REMINGTON: The Science and Practice of Pharmacy, 20th Edition, A. R. Gennaro, ed., (2000). The choice of suitable carriers will depend on the exact nature of the particular vaginal dosage form desired, e.g., whether the chemotherapeutic agent and/or inhibitor of membrane efflux systems is/are to be formulated into a cream, lotion, foam, ointment, paste, solution, microemulsions, liposomal suspension, microparticles, nanoparticles or gel, as well as on the physicochemical properties of the active ingredient(s).

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. In certain embodiments, the pharmaceutical composition is formulated for delayed release of anakinra. In certain embodiments, the pharmaceutical composition is at least partly coated by an enteric-coating agent.

In certain embodiments, the pharmaceutical composition is liquid at room temperature. In certain embodiments, the pharmaceutical composition is semi-solid at room temperature. In certain embodiments, the pharmaceutical composition is solid at room temperature.

In an embodiment, the composition is formulated in accordance with routine procedures as a pharmaceutical composition adapted for oral administration to human beings. In certain embodiments, the formulation comprises lipid-based delivery system and Self-Emulsifying Drug Delivery System (SEDDS).

In an embodiment, the composition is lipid-based delivery system. The composition may comprise water, alcohol such as ethanol, a co-solvent such as propylene glycol or polyethylene glycol, a stabilizer such as butylated hydroxyanisole (BHA) or butylated hydroxytoulene (BHT), a pharmaceutically acceptable sweetener such as sucralose, sucrose, sorbitol or fructose and an anti-oxidant e.g., propyl gallate, lecithin, Vitamin E tocopherol, sesamin, sesamol, sesamolin, alpha tocopherol, ascorbic acid, ascorbyl palmitate, fumaric acid, malic acid, and sodium metabisulphite, disodium EDTA, and combinations of any of the foregoing.

In certain embodiments, the composition is formulated with further pharmaceutically acceptable excipients. Non-limiting examples of such pharmaceutically acceptable excipients include solubilizers for anakinra, stabilizer, bases, preservatives, buffers, viscosity modifiers, bulking agents, gelling agents, emulsifiers, absorption enhancers, surfactants, etc. Further examples of ingredients can found, for example, in the United States Patent or Application Publication U.S. Pat. Nos. 8,222,292B2, 9,345,771B2, and US20090181080A1, hereby incorporated by reference in its entirety.

The SEDDS refers to formulations that are isotropic mixtures of oil, surfactant (with or without co-surfactant) and co-solvent which spontaneously emulsify when exposed to an aqueous medium with gentle agitation. The term “emulsifier” as used herein are amphiphilic molecules that are surface active agents and that stabilize emulsions by reducing the interfacial tension. The term “self-emulsifying” as used herein refers to a composition that forms an emulsion when placed in an aqueous medium. SEDDS have most commonly been studied to improve bioavailability of poorly water soluble drugs via oral administration. The addition of a co-solvent plays a key role in the formation of a self-emulsifying system by significantly reduces the interfacial tension. In so doing, it creates a fluid interfacial film with sufficient flexibility to take up different curvatures required to form microemulsion over a wide range of compositions.

The agents of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In one aspect, the present disclosure provides a dosage form comprising anakinra.

### EXAMPLES

### Example 1: Phase 1/2 Trial for Safety, Tolerability, and Effects of Anakinra

This was a Phase 1/2, eight-week open-label study, followed by a 28-week open-label extension, all of which were preceded and followed by eight-week observational periods.

Eligible participants were age four years and older, with a genetic confirmation of any MPS III subtype. Full inclusion and exclusion criteria are listed below. Potential participants were recruited nationwide with the help of the Cure Sanfilippo Foundation and publicly posted study announcements. Participants were screened in the order that they contacted the study site. Inclusion Criteria were: Mucopolysaccharidosis type III (MPS III) diagnosis confirmed by genetic testing; ≥4 years of age; Patient or parent/legal guardian were able and willing to provide informed consent. For patients 7 to 17 years of age, assent must also have been provided when cognitively possible; If on Genistein, must have been on a stable dose for 6 months prior to enrollment; If on melatonin or other sleep medications, must have been on stable doses for the past 3 months. Also, two of the following criteria were met:
1. Child Sleep Health Questionnaire (CSHQ) Total score≥41.
2. Sanfilippo Behavior Rating Scale (SBRS) Cluster or Domain score≥−2 SD of mean for age group.
3. The presence of significant MPS III related central nervous system (CNS) impairment or behavioral disturbances.
4. Non-communicating Children's Pain Checklist—Revised (NCCPC-R) Total Score of ≥7.
5. Seizure disorder thought to be due to MPS III related disease changes, requiring use of regular medication.
6. Presence of a movement disorder.
   OR, one of the following criteria are met:
1. Previous participation in a gene/cell therapy or enzyme restorative clinical trial.
2. Previous exclusion from a gene/cell therapy or enzyme restorative clinical trial.
3. Functional age as measured by the Vineland (i.e., the Vineland Adaptive Behavior Scales, either the Second or Third Edition) was ≤0.5 chronological age. The Vineland was selected because it has been recommended by two international consensus conferences for assessment of daily functioning in people with MPS disorders, and multiple studies have shown it to be an appropriate measure of functional level as well as sensitive to change in function in MPS III.

Exclusion criteria were: Currently enrolled in another ongoing clinical treatment trial; Previous or current treatment with anakinra, canakinumab or any other IL-1 inhibitor; Use of the following therapies prior to enrollment:
Narcotic analgesics within 24 hours prior to enrollment.
Tocilizumab, dapsone or mycophenolate mofetil within 3 weeks prior to enrollment.
Etanercept, leflunomide, thalidomide, or cyclosporine or intraarticular, intramuscular, intravenous, or oral administration of glucocorticoids within 4 weeks prior to enrollment.
Intravenous immunoglobulin (IVIG), adalimumab, or methotrexate within 8 weeks prior to enrollment.
Infliximab, 6-mercaptopurine, azathioprine, cyclophosphamide or chlorambucil within 12 weeks prior to enrollment.
Rituximab within 26 weeks prior to enrollment;
   Live vaccines within 1 month prior to enrollment; Known presence or suspicion of active, chronic or recurrent serious bacterial, fungal or viral infections, including tuberculosis, HIV infection or hepatitis B or C infection; Clinical evidence of liver disease or liver injury as indicated by presence of abnormal liver tests: AST or ALT>5×ULN, or AST or ALT>3×ULN accompanied by elevated bilirubin>2×ULN; Presence of severe renal function impairment (estimated creatinine clearance<30 mL/min/1.73 m2); Presence of neutropenia (defined as ANC<1200 cells/microliter); History of malignancy; Known hypersensitivity to E. coli-derived proteins, or any components of anakinra; Pregnant or lactating women; Current active infection; History of serious opportunistic infection (e.g., bacterial [Legionella and Listeria]; tuberculosis [TB]; invasive fungal infections; or viral, parasitic, and other opportunistic infections); Positive TB skin test, positive Quantiferon-TB Gold TB test, positive chest xray, or a recent exposure to TB; Requirement for live vaccine exposure that would be expected to occur during the time frame of the study; Any other social or medical condition that the Investigator believes would pose a significant hazard to the subject if the investigational therapy were initiated or be detrimental to the study.

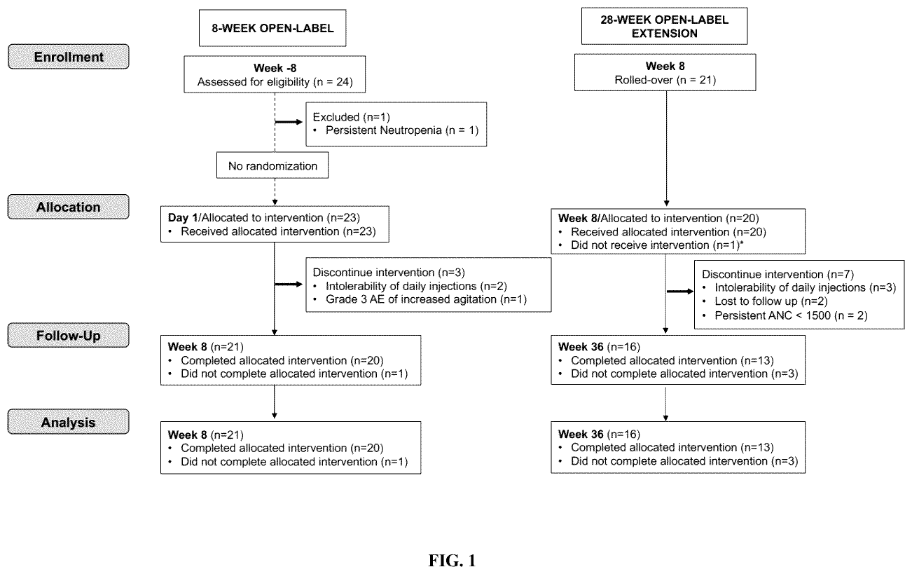

Twenty-four participants (12 males, 12 females) aged six to 26 years were screened and 23 were enrolled as shown in FIG. 1. FIG. 1 is a flow chart for the treatment. Treatment occurred from Day One to Week 36. Observation occurred for eight weeks prior to Day One and for another eight weeks starting at week 36 when treatment was stopped. Primary enrollment target was 20 on treatment at Week Eight. N=3 patients stopped treatment but continued the study by completing assessments through Week 44: one prior to Week Eight (stopped treatment due to a Grade 3 AE of increased agitation), and two between Weeks Eight and 36 (stopped treatment due to persistent ANC<1500).

One participant was excluded due to persistent neutropenia during the screening process. Once enrollment was complete, potential participants more than doubling the study size (N=27) chose to join a waitlist in the event the study was expanded or extended, or future trials were to be developed. Baseline characteristics of enrolled participants are shown in Table 1. Most participants were categorized by their parents/caregivers as white (88%) and not Hispanic (88%). While speaking English was not an inclusion criterion, all caregivers who completed surveys spoke and read English fluently.

TABLE 1
Age- Years

| | |
|---|---|
| Mean ± SD | 10.6 ± 4.2 |
| Median | 9.7 |
| Range | 6.3-26.1 |

Race - no. (%)*

| | |
|---|---|
| White | 21 (88) |
| Black | 1 (4) |
| Asian | 1 (4) |
| Other | 1 (4) |

Ethnicity - no. (%)*

| | |
|---|---|
| Hispanic | 3 (13) |
| Non-Hispanic | 21 (88) |

Sex - no. (%)

| | |
|---|---|
| Female | 12 (50) |
| Male | 12(50) |

MPS Type - no. (%)

| | |
|---|---|
| MPS IIIA | 20 (83) |
| MPS IIIB | 3 (13) |
| MPS IIIC | 1 (4) |

Past Experimental Therapy - no (%)⁺

| | |
|---|---|
| Gene therapy | 3 (13) |
| Enzyme replacement therapy | 3 (13) |

Vineland Adaptive Behavior Scales, 3^{rd}
Edition, Adaptive Behavior Composite ⁺⁺

| | |
|---|---|
| Mean ± SD | 42 |
| Median | 37 |
| Range | 25-69 |

*Race and ethnic group were reported by the participant's parent/caregiver.
⁺Participants were not currently enrolled in another ongoing therapeutic clinical trial.
⁺⁺ Normative population mean ± SD is 100 ± 15. Scores within one standard deviation of the mean, i.e., 85-115, are in the “average Range,” while 70-84 is below average, and <70 is impaired

Five participants withdrew due to intolerability of daily subcutaneous injections (two before Week Eight, three between Weeks 16 and 36), two participants were lost to follow-up, and three were withdrawn by the PI due to AEs, one for a Grade 3 AE and two for persistent neutropenia (<1500 cells/microliter), withdrawn prior to the decision to lower study neutropenia threshold to 1200 cells/microliter. Reasons for intolerability were reported as difficulty keeping the participant still for injections particularly when only one caregiver was routinely available, participant distress in anticipation or delivery of injections, and overall burden of route of administration.

Anakinra was started on Day One at a subcutaneous (SC) daily dose of 100 mg. Dose escalations were made as follows: Either at Week Eight or at Week 16 (if not changed at Week Eight), the daily dose of anakinra was increased to 200 mg SC daily if there was not an improvement from Day One to Week Eight (or week 16) of more than the predefined minimal clinically important difference (MCID, i.e., the smallest improvement considered worthwhile by a patient) in the two outcomes chosen by the parent/caregiver to be “most bothersome” at screening. Of the 20 participants who continued treatment to Week Eight, 12 (60%) required an increased dose of anakinra from 100 mg SC daily to 200 mg SC daily at Week Eight, due to lack of improvement; three additional participants had their dose increased at Week 16. There were no statistically significant differences between those who dose-escalated and those who did not in age, sex, race, or ethnicity, nor efficacy assessments at baseline.

Smaller children with lower body weight need higher dose levels per kilogram body-weight in order to achieve similar anakinra serum exposure as in older, heavier children, due to a higher capacity to eliminate anakinra per kilogram body-weight, based on pharmacokinetic data in 22 systemic-onset juvenile idiopathic arthritis (SJIA) pediatric patients in the age range 2-17 years. As an example, based on this study, a patient with a body weight of 20 kg is predicted to require a dose of 4 mg/kg/day to achieve similar anakinra exposure (area under the curve [AUC]_{0-24h, ss} and C_{min, ss}) as a dose of 100 mg in a 50 kg patient. The patients included in the current study were age 4 years and older. Given that the pharmacokinetics of anakinra in children support a higher dose level per kilogram body weight the same daily dose of 100 mg anakinra was planned for this study. The planned daily anakinra dose 100 mg was within the recommended dose range for anakinra in pediatric patients.

Dose was increased to 200 mg SC once daily, with a maximum dose limit of 8 mg/kg/day, at Week +8 or Week +16 if the change in at least one of the two most bothersome outcomes selected by the parents/guardians at the Day 1 visit had not improved by ≥the minimal clinically important difference (MCID). The exception was the Disordered Movement 7-day Log which did not have a calculatable MCID until the dataset was complete; therefore, dose was increased if there was worsening or no change in either the Duration or Severity as described below. After increase at Week +8, if the MCID was again not achieved after 8 sustained weeks on maximum trial dose of 200 mg (max dose 8 mg/kg/day), treatment with anakinra could be discontinued after a review and discussion between the study PI and subject's parent(s) of their child's individual results from all study outcome measures. For example, at week +8, if a subject had not improved in the chosen items, then the dose was increased to 200 mg SC daily; at week +8, if a subject had improved, then the dose stayed at 100 mg SC daily; however, if this individual then had worsening of the two most bothersome outcomes from week +8 to week +16, after improving from day 1 to week +8, the dose was increased to 200 mg SC daily at that point (i.e., increase at week +16 after not increasing at week +8).

Dose was decreased by 50 mg SC once daily at any time throughout the study if a participant developed any of the following: neutropenia<1200 cells/microliter persistent for ≥2 weeks; thrombocytopenia<50×109 platelets/liter persistent for ≥2 weeks; mild/moderate hypersensitivity reactions including urticaria, rash, and pruritis; CTCAE grade 3 Adverse Event. Anakinra was stopped if the AE above did not resolve within 2 weeks of decreasing the dose by 50 mg SC once daily. Anakinra was restarted at 0.5 times the last dose administered once the AE was resolved. If the AE recured with restarting anakinra, it was discontinued, and the subject monitored per protocol.

### Adverse Events and Safety

The primary safety and tolerability endpoint (Phase 1) was defined as the occurrence of adverse events (AEs) and serious adverse events (SAEs). The primary dosing (Phase 2) endpoint was the percent of participants who required an increase in anakinra dose from 100 mg SC daily to 200 mg SC daily at Week Eight or Week 16. Mean treatment duration was 187±92 days and doses missed were on average 5.2±6.6 doses. Table 2 summarizes adverse events that occurred in ≥5% of participants during treatment with anakinra. Of the 23 participants who received at least one dose of anakinra, 22 (96%) reported at least one adverse event (AE) during the study; most common AEs were injection site reactions, most frequently erythema and swelling, reported in 74% and 43% respectively. There were no unexpected AEs. Three SAEs occurred, none of which were determined to be related to anakinra exposure: two during treatment with anakinra (viral pneumonia and injury [muscle laceration]) and one post-treatment (upper gastrointestinal hemorrhage).

TABLE 2

| | On treatment (N = 23) | | | | Post-treatment* (N = 16) | | | |
|---|---|---|---|---|---|---|---|---|
| | Incidence | | Event rate | | Incidence | | Event rate | |
| Adverse Event | N | % | N | rate | N | % | N | rate |
| Subject with any | 2 | 9% | 2 | 0.09 | 1 | 4% | 1 | 0.04 |
| serious adverse event^{†} | | | | | | | | |
| Subject with any | 22 | 96% | 408 | 17.74 | 15 | 65% | 52 | 2.26 |
| adverse event | | | | | | | | |

Injection site adverse events:

| | | | | | | | | |
|---|---|---|---|---|---|---|---|---|
| Injection site reaction (any) | 18 | 78% | 326 | 14.17 | N/A | N/A | N/A | N/A |
| Injection site erythema | 17 | 74% | 165 | 7.17 | N/A | N/A | N/A | N/A |
| Injection site swelling | 10 | 43% | 52 | 2.26 | N/A | N/A | N/A | N/A |
| Injection site bruising | 5 | 22% | 41 | 1.78 | N/A | N/A | N/A | N/A |
| Injection site itching | 3 | 13% | 65 | 2.83 | N/A | N/A | N/A | N/A |
| Injection site bleeding | 3 | 13% | 3 | 0.13 | N/A | N/A | N/A | N/A |

Non-injection site adverse events:

| | | | | | | | | |
|---|---|---|---|---|---|---|---|---|
| Upper respiratory infection | 7 | 30% | 8 | 0.35 | 4 | 17% | 4 | 0.17 |
| Constipation | 4 | 17% | 5 | 0.22 | 4 | 17% | 4 | 0.17 |
| Agitation | 4 | 17% | 4 | 0.17 | 2 | 9% | 2 | 0.09 |
| Neutropenia | 6 | 26% | 9 | 0.39 | 1 | 4% | 1 | 0.04 |
| COVID19 - mild symptoms | 5 | 22% | 5 | 0.22 | 0 | 0% | 0 | 0.00 |
| Diarrhea | 4 | 17% | 4 | 0.17 | 1 | 4% | 1 | 0.04 |
| Acute otitis media | 2 | 9% | 2 | 0.09 | 2 | 9% | 3 | 0.13 |
| Thrombocytopenia | 4 | 17% | 4 | 0.17 | 1 | 4% | 1 | 0.04 |
| Rash | 1 | 4% | 1 | 0.04 | 2 | 9% | 2 | 0.09 |
| Seizures | 1 | 4% | 1 | 0.04 | 2 | 9% | 3 | 0.13 |
| Stiffness in arms and legs | 1 | 4% | 1 | 0.04 | 2 | 9% | 2 | 0.09 |
| Bronchitis | 2 | 9% | 2 | 0.09 | 0 | 0% | 0 | 0.00 |

*AEs occurring either after early drug discontinuation for participants who continued in the study off treatment and after discontinuation of treatment at week 36 to week 48.
^{†}No treatment related serious adverse events.

### Efficacy

Secondary outcomes were selected to account for the heterogeneity of MPS III symptoms, informed by the caregiver community and consensus recommendations on Sanfilippo trial design.

The Multi-Domain Responder Index (MDRI) was composed of a Sanfilippo-specific behavioral rating scale (SBRS), and standardized ratings of sleep habits (CSHQ), fatigue (PROMIS—Fatigue Parent Proxy Custom Short Form), pain (NCCPC-R), and parenting stress (APSI), as well as parent tracking of disordered movement (Disordered Movement 7-day Log). The MCID, used to determine improvement (change>1 MCID in direction of improvement), worsening, (change>1 MCID in direction of worsening) or no change (change<1 MCID in either direction) on the MDRI, was defined for each of these outcomes based on prior reports in the literature when available or the change during the first 8-week observation period when not available (i.e., disordered movement log). Additional secondary outcomes were mean change in each survey included in the MDRI, described below, followed by a description of the ICR.

For sleep, Child Sleep Health Questionnaire (CSHQ), a paper form, was used. The CSHQ is a parent questionnaire that has been used in many studies to examine both behavioral based and medical based sleep problems in children. The CSHQ yields a total score and eight subscale scores: 1) Bedtime Resistance, 2) Sleep Onset Delay, 3) Sleep Duration, 4) Sleep Anxiety, 5) Night Wakings, 6) Parasomnias, 7) Sleep-Disordered Breathing, and 8) Daytime Sleepiness. It has been validated in multiple groups including community children, children with diagnosed sleep disorders, children with development delay, and children with autism. Higher scores indicate more significant and frequent symptoms. Each item is scored on a scale of 1-3, and a total sleep disturbances score may range from 33-99. MCID was 3.2 based on 469 children aged 4 to 10 years.

For behavior, Sanfilippo Behavior Rating Scale (SBRS) (paper form) was used. The SBRS is a 68 item questionnaire developed to assess the behavioral phenotype of children with MPS III and its progression over time. The SBRS has been validated in Sanfilippo samples and correlated with biomarkers. There are 15 “domain scales” that rate the frequency of symptoms related to orality, movement/activity, attention/self-control, emotional function, and social interaction. In addition, 12 of the 15 domain scales are grouped into four abnormality clusters: Movements, Lack of Fear, Social/Emotional Dysfunction, and Executive Dysfunction. Two domain scales are recommended by the test developers to be analyzed separately: Orality and Mood/Anger/Aggression. Higher scores indicate higher frequency of challenging symptoms. Each item is scored on a scale of 0-6. A mean score is calculated for each SBRS domain and cluster and then an average taken for the SBRS Total score. The mean scores were standardized using the mean and standard deviation from a cohort of MPS III patients, ages 81-220 months. This reference cohort was chosen to best match the age distribution of our participants. MCID (SBRS Total mean score only) was 0.57 based on 18 children with Sanfilippo syndrome aged 6-23 years.

For pain, Non-communicating Children's Pain Checklist—Revised (NCCPC-R) (paper form) was used. The NCCPC-R is a validated scale for measuring pain in children with severe cognitive impairments. It includes 7 scales that measure vocal, social, facial, activity, body and limbs, physiological, and eating/sleeping indicators of pain. A combined total score for pain was calculated as well. Higher scores indicate greater pain. Each item is scored on a scale of 0-3, and a total pain score may range from 0-99. MCID was 4.6 based on 57 non-verbal children aged 3 to 18 years.

For patient fatigue, NIH PROMIS Fatigue-Parent Proxy Custom Short Form (paper form) was used. Per PROMIS guidance, we selected the 10 most relevant questions for parents of children with MPS III from the PROMIS Parent Proxy Fatigue item bank to make a customized PROMIS Fatigue-Parent Proxy Custom Short Form. Customized short forms were scored using an online scoring service. Higher scores indicate more fatigue. Each item is scored on a scale of 1-5, and a total fatigue score may range from 10-50. MCID was =2.2 based on 85 Parents/guardians of children with cancer, sickle cell disease, nephrotic syndrome, or asthma.

For parenting stress, Autism Parenting Stress Index (APSI) (paper form) was used. The APSI was developed based on many interviews of parents of children with autism. It was selected for this study due to findings that many children with MPS III develop autism or autistic symptoms during the course of their disease. The APSI Items fall into three categories: the core social disability, difficult-to-manage behaviors, and physical issues. The APSI measures how much stress the parents are experiencing related to these three categories. The overall APSI scale score has been validated for parents of children with autism and other developmental disabilities. Higher scores indicate greater parenting stress. Each item is scored on a scale of 0-5, and a total APSI score may range from 0-99. MCID was 0.5 based on 139 neurotypical children aged 2 to 6 years.

For disordered Movement, Disordered Movement 7-day Log (paper form) was used. Duration, severity, and type (e.g., dystonia, chorea, other) of movement abnormality was reported by the caregiver in real time for 1 week at a time in a survey. Duration was quantified as: Occasional (<25% of the time)=1; Intermittent (25-50% of the time)=2; Frequent (50-75% of the time)=3; Constant (>75% of the time)=4. Severity was quantified as: The movement has interfered less with my child's daily activities=1; No change in the severity of the abnormal movement=2; The movement has interfered more with my child's daily activities=3. Higher scores indicate increased duration and/or severity of disordered movement. Average was taken of the 7-day average Duration score and the 7-day average Severity score. The total score may range from 1 to 7. MCID was 0.14 based on change in total movement score over first 8 weeks of observation in this study, prior to treatment with anakinra (N=23).

For Individual Clinical Response, Individual Clinical Response (ICR) (paper form) was used. The ICR allowed a caregiver to choose five out of 15 items that they felt were the most impactful. Items chosen based on previous caregiver preference work⁴³ were sleep disturbances, hyperactivity, frustration/impulse control/aggressive behaviors, feeding, anxiety, unhappiness, communication, social deficits, digestive issues and toileting, pain, illness/vulnerability to illness, fatigue, seizure, mobility, and gait. The five items selected by the caregiver were then maintained for longitudinal ratings throughout the trial. The caregiver rated each of these outcomes on a 5-point Likert scale as follows: 0—Not stressful; 1—Sometimes creates stress; 2—Often creates stress; 3—Very stressful on a daily basis; 4—So stressful sometimes we feel we cannot cope. Higher scores indicate more stress, with a possible range of 0-20 for the total ICR at each assessment.

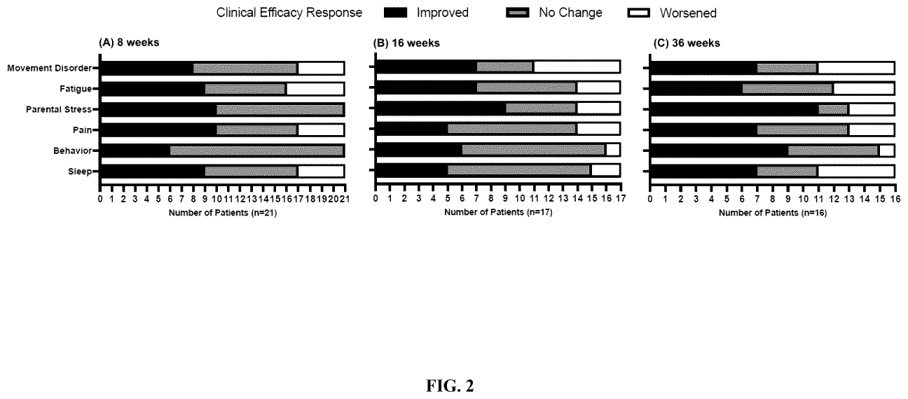

Among above, a multi-domain responder index (MDRI) and an individual clinical response (ICR) were used to capture heterogeneity in treatment response, similar to what has been used previously. Most participants showed improvement beginning at eight weeks of treatment as demonstrated by the MDRI approach for measuring heterogeneous treatment effects as shown in FIG. 2. FIG. 2 shows MDRI for comparison of change during (A) 8 weeks, (B) 16 weeks, and (C) 36 weeks of treatment to minimal clinically important difference (MCID). A change during treatment with anakinra relative to the MCID was used to define “improved”, “no change”, and “worsened”. MCID definitions in Methods, Secondary Outcomes. Specifically, 18 out of 21 (86%) participants improved on ≥1 of the six outcomes included in the MDRI at Week Eight and 15 out of 16 (94%) participants improved on ≥1 of the six outcomes at Week 36. After eight weeks of treatment, improvement of more than the MCID was most common for parenting stress (48%) measured by the Autism Parenting Stress Index (APSI) and pain (48%) measured by the Non-Communicating Children's Pain Checklist—Revised (NCCPC-R). After 36 weeks of treatment, improvement was still most common for parenting stress (69%), but the second most common improvement was behavioral symptoms (56%) measured by the total averaged score on the Sanfilippo Behavior Rating Score (SBRS) followed by pain (44%).

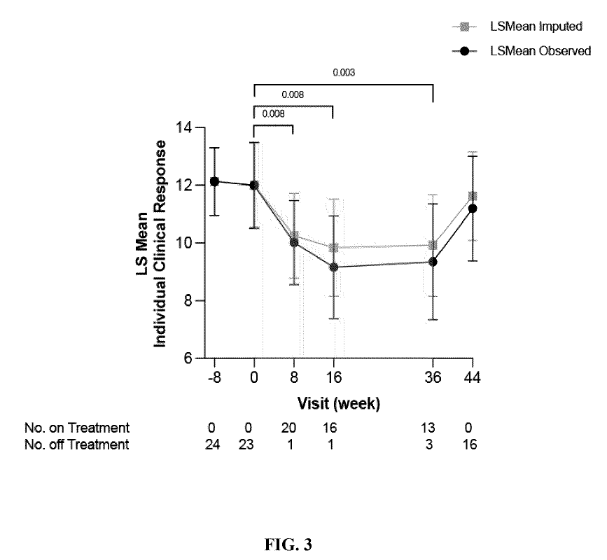

Based on the estimated least-squares means, there was an improvement in the ICR from Day One to Week Eight (−2.0, 95% CI −3.4, −0.6), Day One to Week 16 (−2.8, 95% CI −4.9, −0.8), and Day One to Week 36 (−2.7, 95% CI −4.3, −1.0). A return of symptoms after eight weeks without treatment is demonstrated in the upward slope (i.e., increased severity) between Week 36 to Week 44, as shown in FIG. 3. FIG. 3 shows change in the Individual Clinical Response (ICR) before, during and after treatment with anakinra. Treatment period indicated by grey box (Week Zero to Week 36). The black line graph with circle symbols shows the observed LSMeans with 95% confidence intervals for patients with available data (observed) over time. The imputed LSMeans for the prespecified sensitivity analysis are plotted as grey lines with square symbols. A return of symptoms after eight weeks off treatment, seen by the upward slope to Week 44, suggests the reduction of symptoms from Weeks Zero to 36 were not part of the natural disease course. There was no difference in ICR between the last day of the pre-dosing observational period and the last day of the post-dosing observational period (0.93, 95% CI: −1.18, 3.04).

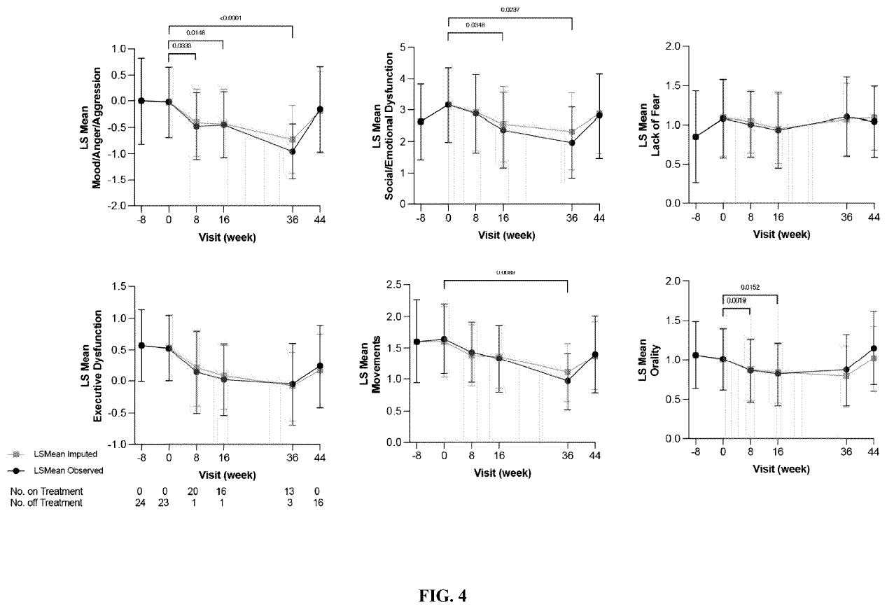

Estimated least-squares means and 95% confidence intervals before, during, and after treatment with anakinra in the SBRS clusters (Movements, Social/Emotional Dysfunction, Lack of Fear, and Executive Dysfunction) and domains (Orality and Mood/Anger/Aggression) are shown in FIG. 4. FIG. 4 shows change in the Sanfilippo Behavior Rating Scale (SBRS) individual clusters and domains before, during and after treatment with anakinra. Treatment period indicated by grey box (Week Zero to Week 36). The black line graph with circle symbols shows the observed LSMeans with 95% confidence intervals for patients with available data (observed) over time. The imputed LSMeans for the prespecified sensitivity analysis are plotted as grey lines with square symbols. P-values are for change in observed LSMeans from Day One. A return of symptoms after eight weeks off treatment, seen by the upward slope to Week 44, suggests the reduction of symptoms from Weeks Zero to 36 were not part of the natural disease course. There was no statistically significant difference in SBRS scores between Week 44 and Day One. There was a positive effect of treatment in all clusters and domains of the SBRS, except for the Lack of Fear and Executive Dysfunction clusters where there was no statistically significant response to treatment with anakinra. On all clusters and domains except for the Lack of Fear cluster, a return of symptoms off treatment is depicted in the upward slope between Week 36 to Week 44, indicating rebounding frequency of symptoms (FIG. 4). There was no difference in any of the SBRS clusters or domains between the last day of the pre-dosing observational period and the last day of the postdosing observational period.

The least-squares mean difference in parenting stress measured by the APSI from Day One to Week 16 was −2.9 (95% CI −5.2, −0.7) and from Day One to Week 36 was −3.8 (95% CI −6.8, −0.8). There were no significant changes in least-squares means for the Child Sleep Health Questionnaire (CSHQ), NIH PROMIS Fatigue-Parent Proxy Custom Short Form, disordered movement 7-day log, and NCCPC-R.

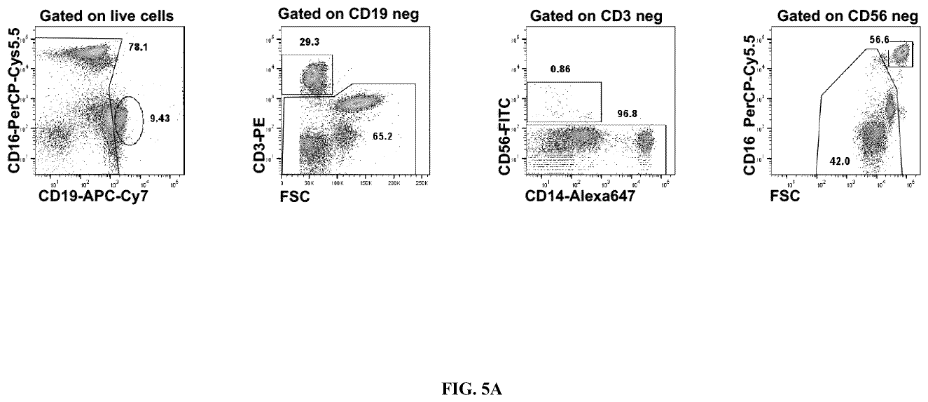

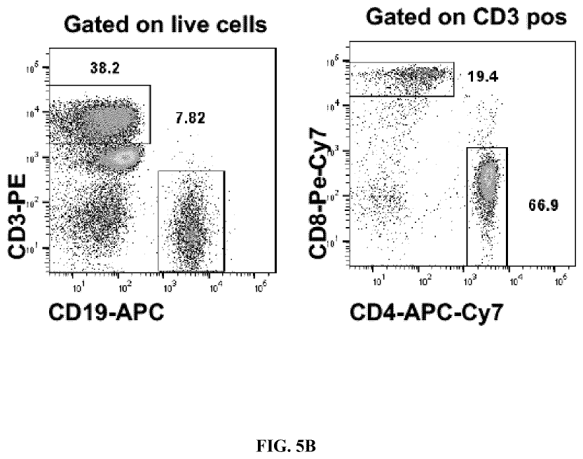

Post hoc analysis to investigate a relationship of the biochemical effects of anakinra with the clinical effects found a statistically significant correlation between an increase in CD4+ T cells and a higher number of improved outcomes in the MDRI (FIGS. 5A-C).

FIGS. 5A-5C shows immunophenotype changes compared to the MDRI. Specifically, FIG. 5A shows a flow cytometry plot of whole blood stained with a specific antibody to identify monocyte. Whole blood cells were gated on live cells then sub-gated on CD19 negative cell (to exclude B cells), CD3 negative cells (to exclude T cells), CD56 (to exclude NK cells). Cells that were not bright CD16 (neutrophils) in the FSC plot were identified as monocyte. These cells expressed CD14 and CD16 (not shown). FIG. 5B shows that B lymphocytes were identified as CD19 positive cells and CD3 negative cells. T lymphocytes CD4 and CD8, were sub-gated from CD3 positive cells. FIG. 5C shows trends for changes in CD4+ T cells (e.g., T helper, T regulatory cells), CD8+ T cells (e.g., cytotoxic T cells), monocytes, and B cells over 16 weeks of treatment with anakinra, compared to the multi-domain responder index (MDRI). Number of MDRI surveys with a treatment response that was either improved (black triangles/solid line) or worsened (open circles/dashed line), as defined by the minimal clinically important difference (MCID), are plotted with immunophenotype data as percent of the cell population. Y-axis is absolute change in percent of the cell population. Positive numbers are an increase and negative numbers are a decrease in cell population. An increase in CD4+ T cells with a decrease in CD8+ T cells and decrease in monocytes is suggestive of a less pro-inflammatory environment. This less pro-inflammatory environment is the desired change with therapy.

These changes coincide with a higher number of improvements on the MDRI outcomes. Anakinra is not expected to have a significant effect on B cells. The lack of B cell change and lack of relationship with MDRI outcomes may increase confidence in the findings. Although correlations with other cell types in this small sample did not reach statistical significance, in general, the less pro-inflammatory the environment (the desired effect, i.e., an increase in CD4+ T cells and decreases in CD8+ T cells and in monocytes), the more the MDRI outcomes showed improvements (FIG. 4). In contrast, MDRI outcomes that showed worsening were associated with more pro-inflammatory markers, i.e., decrease in CD4+ T cells, and increases in CD8+ T cells and in monocytes.

To summarize, this study indicated anakinra is safe for further investigation while curative therapies are in development. Further, this study showed clinically meaningful improvements in various neurobehavioral and quality of life-related outcomes, suggesting that an anti-inflammatory approach might prove to be symptom-modifying for Sanfilippo syndrome.

No new safety concerns were identified. All adverse events were mild or moderate and no serious adverse events were considered to be related to anakinra. On the other hand, 60% of participants increased anakinra dose to 200 mg SC daily at week 8-85% of participants who completed week 36 were receiving 200 mg SC daily. Also, one or more parent/caregiver reported behavioral and somatic symptoms improved in most participants treated with anakinra: 86% of participants improved in at least one outcome at week 8; and 94% of participants improved in at least one outcome at week 36. Further, Movement, Social/Emotional Dysfunction, Mood/Anger/Aggression, and Orality had a statistically significant improvement during treatment with anakinra.

Accordingly, the results from this phase 1/2 study provide evidence that targeting inflammation through use of anakinra is safe and may improve neurobehavioral symptoms and meaningful aspects of the lived experience of patients and families affected by Sanfilippo syndrome.

### Example 2: Phase 1/2 Trial for Safety, Tolerability, and Effects of Anakinra

Subjects from Example 1 were further studied for effects of anakinra on sleep problems, loose stool, and behavioral problems for subjects having Sanfilippo syndrome. For the current analysis, there were 14 patients in the Sleep Problems Cohort, 13 in the Loose Stool Cohort, and 20 in the Behavioral Problems Cohort. Baseline demographics of each symptom-specific subgroup are shown in Table 1 in Example 1.

All eligible participants were four years and older in age with a genetic confirmation of MPS III. Participants were recruited nationwide with the help of Cure Sanfilippo Foundation and publicly posted study advertisements including on clinicaltrials.gov. The dose of anakinra was increased to 200 mg subcutaneous daily by week 16 in all participants who continued on treatment through week 36.

### Symptom-Specific Subgroup Population

From the screened study population, three subgroups were created based on specific cutoff scores on corresponding outcomes. Participants with a Children's Sleep Habits Questionnaire (CSHQ) total score>40 were included in the “sleep disturbances” subgroup. This cut-off score of >40 was based on a prior publication showing a sensitivity of 0.80 and specificity of 0.72 for identifying children with a sleep disorder. The following criteria was used to define clinically significant symptoms: participants who marked at least one ‘3—interfered quite a bit’ or two ‘2—interfered somewhat’ in the four questions related to loose stool in the Sanfilippo Stool Habits Questionnaire (SSHQ) were included in the “loose stooling” subgroup. For the “behavioral problems”, participants that either scored one ‘3—(About half the time)’, two ‘2—sometimes (25% of time) or a total score≥4 in the three questions corresponding to the mood/anger items, or scored at least one ‘2—sometimes (25% of time)’ or two or more ‘1—occasionally (5-10% of time)’ in the four questions corresponding to the aggression items of the mood/anger/aggression cluster of Sanfilippo Behavior Rating Scale (SBRS) were added to the “behavioral problems” subgroup.

To determine the MCID of both the SSHQ loose stool severity, and SBRS mood/anger/aggression, the standard deviation scores at baseline from our specific population were multiplied by an effect size of 0.5, due to specificity of the survey not being available.

### Sleep Disturbances

The Children's Sleep Habits Questionnaire (CSHQ) total score and total number of sleep problems on the CSHQ was used to measure effect of anakinra on sleep disturbances. The CSHQ is a parent reported questionnaire that has been used to examine behavioral-based and medical-based sleep problems in children. It has been validated in multiple groups, including community children and children diagnosed with sleep disorders, development delay and/or autism. Higher scores represent more significant and/or frequent symptoms. Each item for the total score is scored on a scale of 1-3 and a total sleep disturbances score can range from 33 to 99. Total sleep problems range from 0 to 33.

Minimal clinically important difference (MCID, i.e., the smallest improvement considered worthwhile by a patient) was 3.72 based on 469 children aged 4-10 years for the CSHQ total, and MCID was 4.19 for the total Sleep Problems, based on an effect size of 0.5 in our selected population.

### Loose Stooling

Change in the Sanfilippo Stool Habits Questionnaire (SSHQ) loose stool severity total score was used to examine the effects of anakinra on diarrhea in this subgroup. The SSHQ uses a caregiver proxy to report on individuals with significantly limited communication abilities due to medical conditions, intellectual disability or age. Questions cover frequency and severity of loose stools, along with impact on daily activities (i.e., attending daycare, school, or other learning center, and family outings) and behavior. Higher scores represent increased severity in loose stooling. Each of the give items for the total loose stool score is scored on a scale of 0-3 and a total loose stool score can range from 0 to 15.

MCID was 2.37 based on our selected population, and an effect size of 0.2, since reliability was not available for the SSHQ.

### Behavioral Problems

Change in the Sanfilippo Behavior Rating Scale (SBRS) mood/anger/aggression cluster was used to examine the effects of anakinra on behavioral problems in this subgroup. The SBRS is a 68-item questionnaire that was developed to assess the behavioral phenotype of children with Sanfilippo syndrome and its progression over time. The mood/anger/aggression cluster contains questions related to the severity of the child's mood, self-injurious behavior, and aggression towards other people and/or inanimate objects. Higher scores indicate higher frequency of symptoms. Each item is scored on a scale of 0-6 and a total mood/anger/aggression score can range from 0 to 42.

MCID was 0.41 based on reliability score of 0.832 from 18 children with Sanfilippo syndrome aged 6-23 years.

### Statistical Analyses

A repeated measures analysis of variance (ANOVA) was used to assess changes over time, with time serving as the within-subject factor. This analysis used an autoregressive (AR (1)) covariance structure for the repeated measures and presented the least-squares means along with 95% confidence intervals (CI). Residual analyses, including studentized and Pearson residuals, were done and compared to the standard normal quantile-quantile plot to assess the model's adherence to its underlying assumptions, and no serious violations were detected. Data were analyzed using SAS version 9.4 (SAS Institute). All tests were two-sided, and a p-value<0.05 was considered statistically significant.

### Results

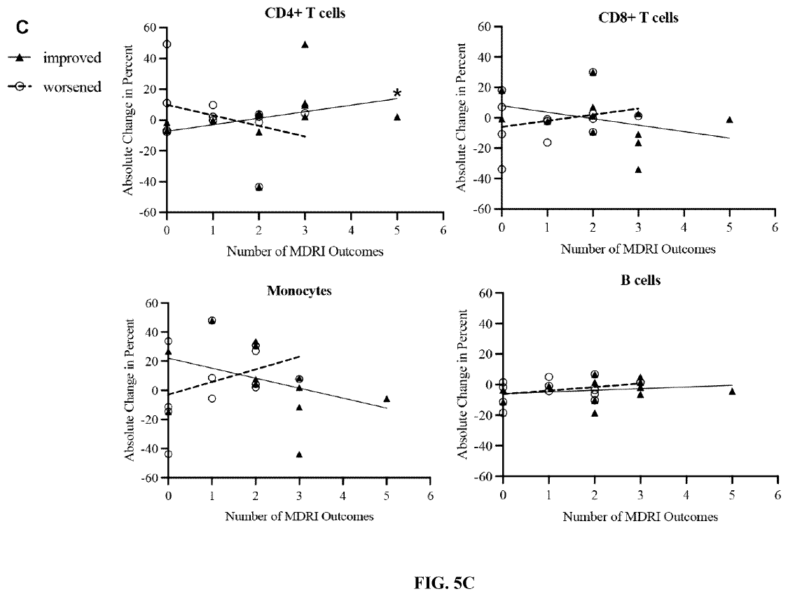

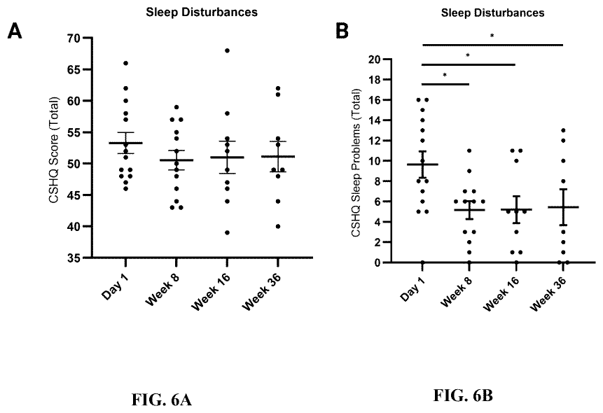

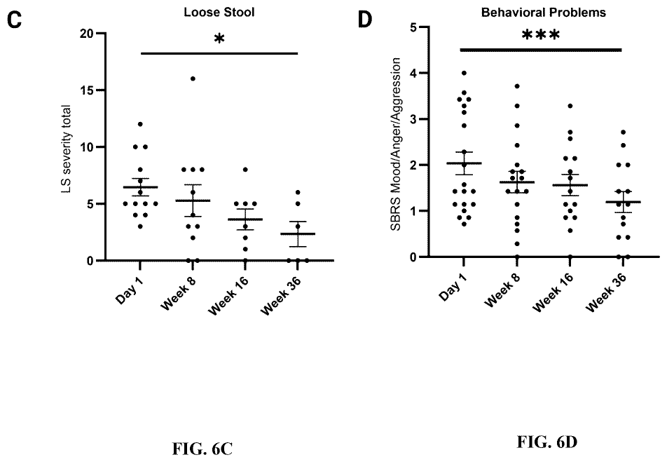

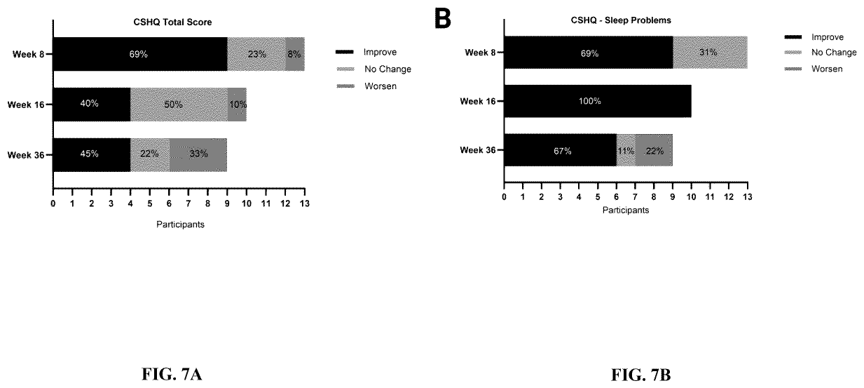

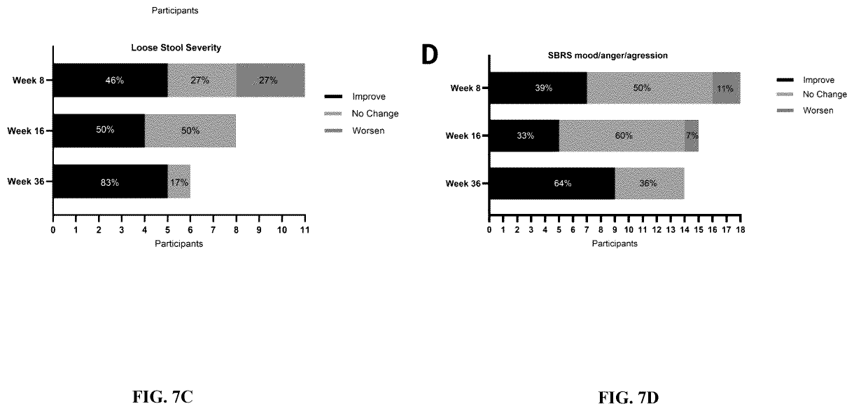

FIGS. 6A-6D shows CSHQ total score in “Sleep Disturbances” subgroup (FIG. 6A), total number of Sleep Problems in the CSHQ in “Sleep Disturbances” subgroup (FIG. 6B), LS severity (FIG. 6C) in “Loose Stool” subgroup, and SBRS mood/anger/aggression in “Behavioral Problems” subgroup (FIG. 6D) at Day 1, Week 8, Week 16, and Week 36. FIGS. 7A-7D shows minimal clinically important difference (MCID) changes in functional outcomes and behavior after anakinra treatment.

As shown in FIG. 6A, there was no significant effect of anakinra on the CSHQ total score in participants in this subgroup, compared to baseline. However, anakinra significantly improved the total number of Sleep Problems in this subgroup at Week 8 (5.34, 95% CI: 2.66, 8.02), Week 16 (5.44, 95% CI: 2.45, 8.43) and Week 36 (6.13, 95% CI: 3.03, 9.23), compared to baseline, as shown in FIG. 6B, so there was a significant reduction in the total number of Sleep Problems after eight, 16 and 36 weeks of treatment.

Using a minimal clinically important difference (MCID) to define response as “improved”, “no change” or “worsened”, at Week 8, 69% of participants (n=9) improved on the CSHQ total score, 23% (n=3) had no change, and 8% (n=1) worsened, as shown in FIG. 7A. At Week 16, 40% (n=4) improved, 50% (N=5) had no change, and 10% N=1) worsened. At 36 weeks, 45% (n=4) improved, 22% (n=2) had no change, and 33% (n=3) worsened. For the total number of Sleep Problems, at Week 8, 69% (n=9) improved, 31% (n=4) had no change, and 0% worsened, as shown in FIG. 7B. At Week 16, 100% (n=10) improved and at Week 36, 67% (n=6) improved, 11% (n=1) had no change, and 22% (N=2) worsened.

As shown in FIG. 6C, in participants with loose stooling problems at baseline, anakinra improved loose stool severity scores after 36 weeks of treatment. Stooling significantly improved after 36 weeks of anakinra (2.78, 95% CI: −0.12, 5.68). Using the MCID approach, at Week 8, the loose stool score improved in 46% (n=5), 27% (n=3) had no change, and 27% (N=3) worsened, as shown in FIG. 7C. At Week 16, 50% (n=4) had an improved score and 50% (n=4) had no change, none worsened. By 36 weeks, 83% (n=5) improved and 17% (n=1) had no change in the loose stool score; again, none worsened. Given anakinra's anti-inflammatory mechanism and the observed improvement in loose stooling with treatment, it is contemplated that intestinal inflammation contributes to loose stool symptoms in Sanfilippo syndrome.

As shown in FIG. 6D, there was a significant improvement in the SBRS mood/anger/aggression domain in participants who met the criteria for our “behavioral problems” subgroup. Anakinra significantly improved SBRS mood/anger/aggression scores at Week 36 (1.28, 95% CI: 0.72, 1.85). Similarly, when comparing individual changes in the SBRS mood/anger/aggression cluster score to a MCID, after eight weeks of anakinra 39% (n=7) had an improvement, 50% (n=9) had not change, and 11% (n=2) worsened, as shown in FIG. 7D. At Week 16, 33% (n=5) improved, 60% (n=9) had no change, and 8% (n=1) worsened. By 36 weeks, 64% (n=9) improved, 36% (n=5) had no change, and none worsened. As previously mentioned, behavioral problems are very challenging in this population. Understanding that this is a critical symptom to be treated for the families, it is very encouraging for us to observe statistically and clinically meaningful changes to behavioral problems following anakinra treatment.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

What is claimed is:

1. A method for treating a mucopolysaccharidosis III (MPS III) in a patient in need thereof, comprising administering to the patient an effective amount of a polypeptide comprising human IL-1Ra or a sequence having at least 85% sequence identity to the human IL-1Ra.

2. The method of claim 1, wherein the polypeptide comprises anakinra.

3. The method of claim 1, wherein the polypeptide consists of anakinra.

4. The method of claim 1, wherein the polypeptide is administered at 50 mg to 500 mg daily.

5. The method of claim 1, wherein the polypeptide is administered at 100 mg or 200 mg daily.

6. The method of claim 1, wherein the polypeptide is administered once daily.

7. The method of claim 1, wherein the administration is subcutaneous.

8. The method of claim 1, wherein the polypeptide is administered for 8 weeks or more.

9. The method of claim 1, wherein the polypeptide is administered for 16 weeks or more.

10. The method of claim 1, wherein the polypeptide is administered for 36 weeks or more.

11. The method of claim 1, comprising administering 100 mg or 200 mg of anakinra once daily.

12. The method of claim 1, wherein the MPS III is MPS IIIA, MPS IIIB, MPS IIIC, or MPS IIID.

13. The method of claim 1, wherein the MPS III is MPS IIIA, MPS IIIB or MPS IIIC.

14. The method of claim 1, wherein the patient has at least one of: a movement disorder; fatigue, pain, a Sanfilippo behavior, a sleep problem, loose stool, hyperactivity, communication problems, or behavioral problem.

15. The method of claim 1, wherein the patient has a child Sleep Health Questionnaire (CSHQ) Total score≥41.

16. The method of claim 1, wherein the patient has a Sanfilippo Behavior Rating Scale (SBRS) Cluster or Domain score≥−2 SD of mean for age group.

17. The method of claim 1, wherein the patient has significant MPS III related central nervous system (CNS) impairment or behavioral disturbance.

18. The method of claim 1, wherein the patient has a Non-communicating Children's Pain Checklist—Revised (NCCPC-R) Total Score of ≥7.

19. The method of claim 1, wherein the patient is an adult (18 years or older).

20. The method of claim 1, wherein the patient is a minor (17 years or younger).
